(19) United States
(12) Patent Application Publication
(10) Pub. No.: US 2017/0189573 A1
(43) Pub. Date: Jul. 6, 2017

(54) NOVEL HEMOSTATIC PATCH AND USES THEREOF

(71) Applicants: Leo RUBIN, Suffern, NY (US); Shaker MOUSA, Wynantskill, NY (US)

(72) Inventors: Leo RUBIN, Suffern, NY (US); Shaker MOUSA, Wynantskill, NY (US)

(21) Appl. No.: 15/405,202

(22) Filed: Jan. 12, 2017

Related U.S. Application Data
(63) Continuation of application No. 14/949,715, filed on Nov. 23, 2015, now abandoned.
Application No. 14/949,715 is a continuation of application No. 14/236,815, filed on May 27, 2014, now abandoned.
(60) Provisional application No. 61/514,587, filed on Aug. 3, 2011.

(51) Int. Cl. A61L 15/28; C08B 37/08; A61L 15/64; A61L 15/22; A61L 15/44; A61L 15/58

(52) U.S. Cl. CPC A61L 15/28; A61L 15/44; A61L 15/58; A61L 15/64; A61L 15/225; C08B 37/003; A61L 2300/204; A61L 2300/418; A61L 2300/62; A61L 2400/04; A61L 2300/252

(57) ABSTRACT

Disclosed herein is a novel hemostatic patch that may be used to control and/or arrest bleeding in patients. The patch offers an effective but also minimally invasive way to control and/or arrest bleeding in a patient. The patch comprises a mucoadhesive and a compound that causes vasoconstriction. In a preferred aspect, the patch comprises chitosan and Neuropeptide Y. Also disclosed are methods of using the novel hemostatic patch.

### CROSS REFERENCE TO RELATED APPLICATIONS

This application claims the benefit of U.S. Provisional Application 61/514,587 filed Aug. 3, 2011, the contents of which are incorporated herein by reference.

### TECHNICAL FIELD

The field relates generally to a hemostatic device. The field further relates to methods for using said hemostatic device. The field further relates to a hemostatic patch that comprises both a mucoadhesive and a compound that causes vasoconstriction.

Specifically, the field relates to a hemostatic patch that comprises chitosan and a vasoconstrictant, e.g., neuropeptide Y, epinephrine.

### BACKGROUND OF THE INVENTION

Despite the products that have been made available, and the techniques now known, the control or arrest of patient bleeding, by virtue of the most minimally invasive means achievable, still remains a unique and salient issue for many physicians. In particular, physicians still seek some comprehensive, effective, and minimally invasive means in order to treat patient bleeding where said bleeding arises due to internal organ or tissue trauma, or alternatively arising because of vascular complications associated with catheterization.

For example, internal tissue wounds present unique problems that must be addressed when attempting to close such wounds. Where there is bleeding within the field of injury around the wound, consequently this can cause the wound to be difficult to locate. The time lost from treating the actual point of trauma can, in some instances, be life-threatening depending upon the amount of blood loss a patient may incur as a result of lost time. Moreover, because access to an internal wound sometimes requires open surgery this can necessarily cause risk to the patient. Moreover, certain surgical instruments may be called upon to perform tasks with the aid of other instruments in order to control or arrest bleeding suffered by a patient. However, consistent and reliable closure of a wound using only a single instrument is difficult to achieve.

Furthermore, localized vascular complications associated with catheterization of a vein or an artery is often times the common side effect of many medical procedures. Such complications including hemorrhaging of blood vessels, delayed homeostasis time, hematoma, pseudoaneurysm, and arteriovenous (AV) fistula formation can be life threatening. Since cardiac catheterization, for example, remains the primary technique for diagnosing coronary artery disease and is used to help delineate coronary anatomy, the information gained is often a medical necessity, despite the risks. Traditionally, the application of pressure to a vein or artery after cardiac catheterization can be very painful for the patients. Compression bandages with weights may be applied for 4-8 hours following hemostasis and patients can be immobilized up to 24 hours. However, patients are then at risk to develop back pain and urinary retention in addition to the localized vascular complications.

One problem with certain presently available hemostatic patches occurs because, while they may be effective at the interface of the wound and the patch, hemostatic patches have not been demonstrated to be effective at a distance in treating, for example, punctures caused by catheterization that may arise due to damage or trauma that arise from deeper internal puncture in the vein or artery.

The products in primary use in many cases, for wound closure, are surgical sutures and staples. Sutures are recognized to provide adequate wound support. However, sutures may cause additional trauma to the wound site and are time consuming to replace. Surgical staples have also been developed to speed wound apposition and provide improved cosmetic result. However, surgical staples may also impose additional wound trauma and require the use of ancillary and often expensive devices for positioning and applying the staples. In addition, it may be the case where serious bleeding exists, for example, that endoscopically stapling a wound does not function to arrest arterial bleeding. Therefore, because the wound continues to bleed even after the endoscopic insertion of a staple, open surgery may still be required. Such surgery is necessarily more invasive, and may be more painful and entail a greater risk of infection than if a physician were able to simply arrest bleeding at the point of injury through some other less invasive means.

Therefore, there still remains a very pertinent and immediate need for a means of controlling and/or arresting patient bleeding that is safe, consistently effective, and minimally invasive to the patient.

### BRIEF DESCRIPTION OF THE FIGURES

FIG. 1 depicts a flow chart diagram for the synthesis of chitosan nanoparticles encapsulating NPY.

FIGS. 2A and 2B depict flow charts for the synthesis of chitosan PLGA-nanoparticles.

FIGS. 3A and 3B depict flow charts for the synthesis cross-linked nanoparticles encapsulating NPY.

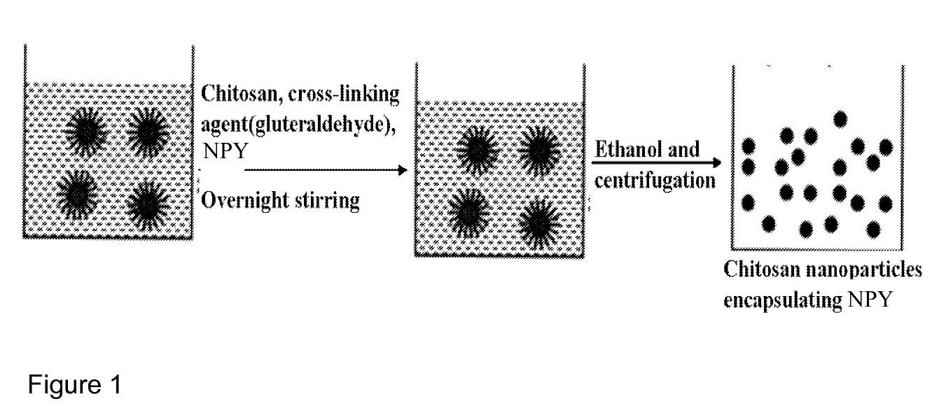

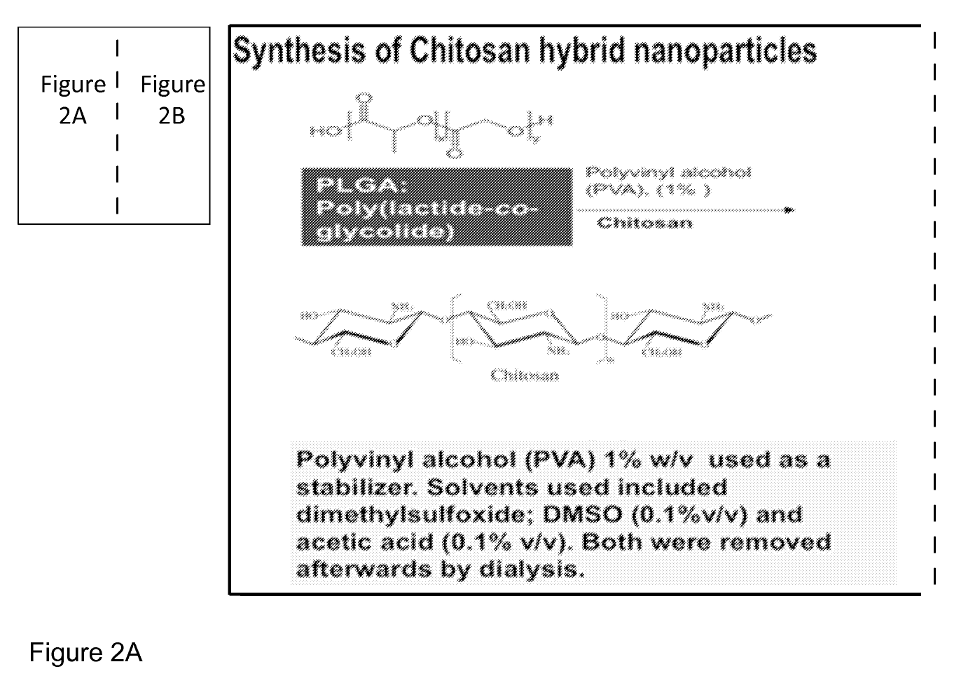

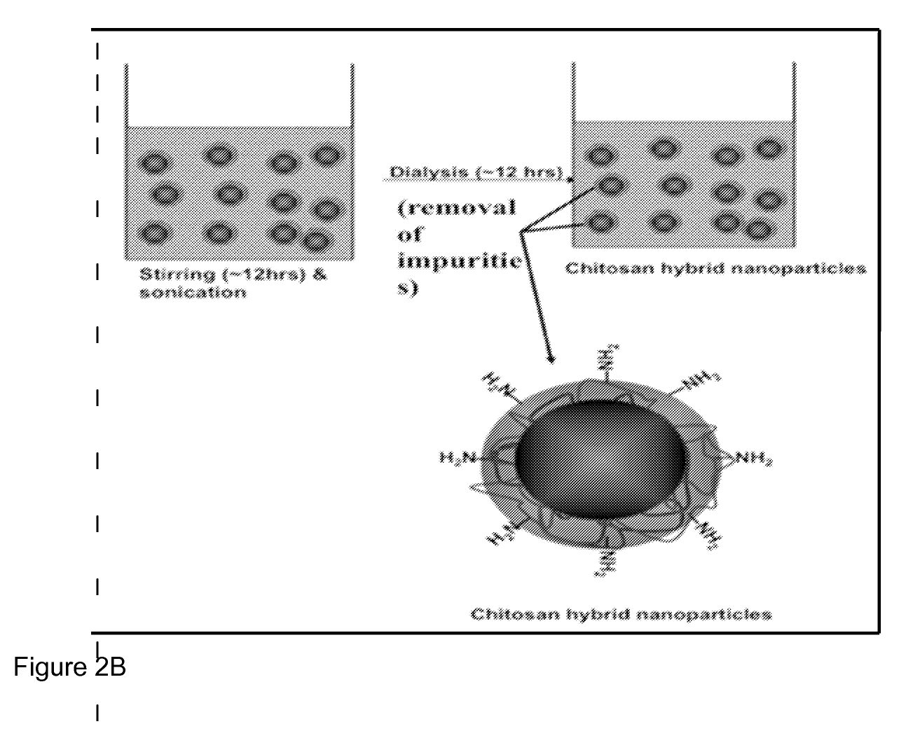

### SUMMARY OF THE INVENTION

Chitosan derives from chitin, an abundant natural polymer commonly found in the exoskeletons of crustacean and insects as well as the cell walls of fungi. Chitosan is composed of glucosamine and N-acetyl glucosamine, which are linked in a beta (1-4) manner. The molecular weight and degree of acetylation, which are critical in determining the characteristics of chitosan, depend on the source and production process used. An intriguing and important feature of chitin-based material is its cationic nature. In many physiological situations, chitosan can become protonated and positively-charged. The positive charge of the chitosan originates from the protonated amino groups. Protonated chitosan can form a complex with many types of negatively-charged molecules, such as growth factors, nucleic acids, and cytokines. This feature allows chitosan to recruit and bind bioactive factors from surrounding environments, thereby protecting these factors from degradation and increasing local concentration and efficacy. This unique property of chitin-based materials is significant in the modulation of cell behavior during tissue regeneration. (Yang, Int J Mol Sci., 12(3): 1936-63 (2011))

Neuropeptide Y (NPY) is a 36-amino acid peptide neurotransmitter that is located throughout the central and peripheral nervous systems. (Tatemoto (1982) Proc. Natl. Acad. Sci. USA 79: 5485). It affects a broad range of phenomena, including blood pressure regulation, memory, anxiolysis/sedation, food and water appetite, vascular and other smooth muscle activity, intestinal electrolyte secretion, and urinary sodium excretion. Neuropeptide Y is a compound capable of potent vasoconstriction.

The present invention provides for a hemostatic patch which functions to control and/or arrest bleeding in a patient. In one aspect, the hemostatic patch comprises a mucoadhesive compound in combination with a compound that causes vasoconstriction. In a further aspect, hemostatic patch comprises chitosan and a vasoconstrictant. In one aspect the hemostatic patch comprises nanoparticles further comprising chitosan and at least one vasoconstrictant, e.g. Neuropeptide Y, epinephrine, norepinephrine, vasopressin, phenylephrine (e.g., NEO-SYNEPHRINE®), pseudoephedrine, metaraminol (e.g., ARAMINE®). In one aspect the hemostatic patch comprises one or more of vasoconstrictors in combination with one another.

In one aspect, it is contemplated that the mucoadhesive compound, and the compound that causes vasoconstriction, are both present in a stable matrix. In still another aspect, it is contemplated that the mucoadhesive compound, and the compound that causes vasoconstriction, are both linked by covalent bonding.

In one aspect of the present invention the hemostatic path comprises a mucoadhesive compound that is chitosan and a compound that causes vasoconstriction which is Neuropeptide Y (“NPY”), wherein both are present in a stable matrix. In yet another aspect the NPY is covalently bound to the chitosan. In a further aspect the hemostatic patch comprises chitosan and epinephrine wherein both are present in a stable matrix. In another aspect the hemostatic patch comprises chitosan and epinephrine wherein epinephrine is covalently bound to the chitosan.

### DETAILED DESCRIPTION

“Mucoadhesive compound”, or “mucoadherent”, refers to a compound or agent that adheres themselves, or causes adhesion of another compound to a mucosal surface, e.g., chitosan, chitosan salts, or chitosan derivatives. Mucoadhesive compounds can be considered those compounds which also increase the amount of contact time of a compound or matter to a mucosal layer.

“Compound that causes vasoconstriction”, “vasoconstrictant”, “vasoconstrictor”, or similar terms as used herein refer to both exogenous and endogenous vasoconstrictors. Endogenous vasoconstrictors may include, e.g., Neuropeptide Y, Peptide YY, Angiotensin II, Muscarinic agonists, Endothelin and Andrenergic agonists. “Vasoconstrictor”, “Vasoconstrictant”, and/or “Compound that causes vasoconstrictions” as used herein may refer to e.g., derivates, analogues, like compounds, or salts, of said vasoconstrictors.

“Effective amount”, as used herein refers to the amount of a compound necessary to control or arrest bleeding in a patient. One of ordinary skill in the art would appreciate the amounts necessary to effectuate treatment.

“Patch”, “hemostatic patch”, or “device” as used herein refers to bandages, patches, powders, gels, sponges, or any other type of scaffold known in the art that could be used as a scaffold for the composition comprising a mucoadhesive compound and vasoconstrictant.

“Chitosan” as referred to herein may also encompass chitosan and chitosan derivatives and/or chitosan salts, e.g., chitosan acetate, chitosan lactate, chitosan formate, chitosan maliate, chitosan chloride, chitosan ascorbate, or chitosan citrate.

The present invention contemplates that the hemostatic patch comprises an effective amount of a mucoadhesive and an effective amount of a compound that causes vasoconstriction either in a stable matrix and/or covalently bonded to one another and/or to a cross-linker.

In one aspect, the present invention contemplates that Neuropeptide Y may refer to Neuropeptide Y derivatives and like compounds. For example, in one aspect the present invention contemplates that Peptide YY can be used in combination with a mucoadhesive compound, e.g., chitosan and/or chitosan salts and/or derivatives. In one aspect the invention is directed to a hemostatic patch that comprises chitosan salts and/or chitosan derivatives and a compound that causes vasoconstriction. It is contemplated by the present invention that the hemostatic patch comprises chitosan salts and/or chitosan derivatives and Neuropeptide Y.

In another aspect, the present invention also contemplates that a mucoadhesive compound other than chitosan may be utilized. For example, in one aspect the present invention contemplates that hydrogels other than chitosan may be used in combination with the hemostatic patch. Hydrogels other than chitosan may be selected from hydrogels based on natural materials, e.g., collagen, starch, gelatin, alginates, and/or dextrans. In one aspect, hydrogels other than chitosan may also be selected from hydrogels based on synthetic polymers, e.g., N-vinylpyrrolidone, poly(vinyl alcohol), polyphosphazenes, poly(ethylene oxide)-b-poly(propylene oxide), or copolymers. In one aspect, the present invention contemplates that the hemostatic patch comprises mucoadhesives, other than chitosan, in combination with chitosan either alone or in combination with a vasocontrictor, e.g., Neuropeptide Y, Neuropeptide Y analogues, epinephrine, norepinephrine, vasopressin, phenylephrine (e.g., NEO-SYNEPHRINE®), pseudoephedrine, metaraminol (e.g., ARAMINE®).

In one aspect, the present invention provides Hemostatic Patch A which comprises vasoconstrictor nanoparticles, wherein the vasoconstrictor is encapsulated or immobilized by a bioabsorbable polymer, and wherein said nanoparticles comprise at least one vasoconstrictor, e.g., a vasoconstrictor selected from any of the following:

1. Neuropeptide Y

2. Neuropeptide Y analogues

3. epinephrine

4. norepinephrine

5. vasopressin

6. phenylephrine (e.g., NEO-SYNEPHRINE®)

7. pseudoephedrine, metaraminol (e.g., ARAMINE®)

8. Peptide YY

9. Angiotensin II

10. Muscarinic agonists

11. Endothelin; and/or

12. Andrenergic agonists

Wherein the vasoconstrictor is encapsulated or immobilized on a bioabsorbable polymer, e.g. having any of the characteristics of a.-s. In one further aspect the bioabsorbable polymer is chitosan, e.g., having any of the characteristics of herein disclosed list k.-s.

In one aspect, the present invention provides Hemostatic Patch B which comprises vasoconstrictor nanoparticles, wherein at least one vasoconstrictor, e.g., any of the vasoconstrictors listed in the foregoing list numbered 1-12, is encapsulated or immobilized by a bioabsorbable polymer, and, for example, wherein the bioabsorbable polymer has any of the following characteristics:

a. Wherein the polymer comprises chitosan.

b. Wherein the polymer comprises poly(lactic-co-glycolic acid) (PLGA) or polylactic acid (PLA), e.g., PLGA having 50/50 co-polymerization of D,L-lactic acid and glycolic acid.

c. Wherein the polymer comprises chitosan crosslinked using glutaraldehyde.

d. Wherein the polymer comprises chitosan linked to bile acids.

e. Wherein the polymer comprises chitosan linked to PLGA, e.g., using glutaraldehyde as crosslinker.

f. Any of the foregoing wherein the vasoconstrictor nanoparticles have an average diameter of about 50-1000 nm, e.g., 50-500.

g. Any of the foregoing wherein the vasoconstrictor nanoparticles have an average diameter of about 100-300 nm, e.g., 200 nm.

h. Any of the foregoing wherein the nanoparticles have a zeta potential of 0-100 mV, e.g., 50-100 mV or 0-50 mV.

i. Any of the foregoing wherein the nanoparticles have a zeta potential of 10-20 mV, e.g., 20 mV.

j. Any of the foregoing wherein the nanoparticle comprises a second pharmacologically active ingredient.

In one aspect, the present invention provides Hemostatic Patch C which comprises a vasoconstrictor nanoparticle, wherein at least one vasoconstrictor, e.g. selected from any of the foregoing list numbered 1-12, is covalently linked to the bioabsorbable polymer e.g., having any of the charactistics of foregoing list a.-j. For example, in one specific example the vasoconstrictor is NPY which is linked via an activated linker group, e.g., an epoxide moiety, for example a moiety capable of coupling to an amine group on the bioabsorbable polymer, for example the amino moieties on chitosan. Similar methods and chemistry of linking are known in the art.

In one aspect, the present invention provides Hemostatic Patch D which comprises vasoconstrictor-nanoparticles, wherein the nanoparticles comprise a bioabsorbable polymer, e.g. having the characteristics of any of foregoing list a.-j. , and at least one vasconstrictor, e.g., selected from any of the vasoconstrictors in the foregoing list numbered 1-12, wherein the vasoconstrictor is encapsulated or immobilized on a bioabsorbable polymer and the nanoparticles are made from any the following components:

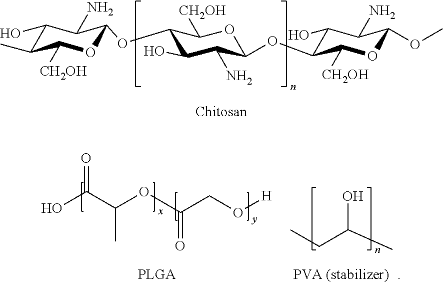

In one aspect, Hemostatic Patch E comprises nanoparticles comprise a bioabsorbable polymer, e.g. having any of the characteristics of foregoing list a.-j., and a vasoconstrictor, e.g. selected from any of the foregoing list 1.-12., wherein the hemostatic patch has these components in approximately the following amounts:

| | Approx Amount | |
|---|---|---|
| Components | (% w/w) | |
| of the | in the | |
| formulation | nanoformulation | Role in the formulation |
| Chitosan | 50-70%, e.g. 60% | Component of the nanocarrier |
| PLGA | 20-30%, e.g. 25% | Component of the nanocarrier |
| Vasoconstrictor, e.g., | 10-20%, e.g. 15% | Active ingredient (chemically |
| of List Numbered 1-12 | | conjugated to the |
| (e.g. NPY, | | nanoparticles) |
| epinephrine) | | |

The contents of the nanoparticles are confirmed using, e.g. HPLC and LC/MS. The nanoparticle formulations may be sterilized using conventional means, e.g., filtration, gamma radiation.

As noted, in one aspect the present invention contemplates that the hemostatic patch comprises both a bioabsorbable polymer, e.g., having the charactistics of foregoing list a.-j., and a vasoconstrictor, e.g. of foregoing list 1-12. In a specific aspect the present invention comprises chitosan and NPY both of which are present in a stable matrix. In yet another specific aspect, the present invention comprises chitosan and Neuropeptide Y linked by covalent bonding.

In one aspect, the present invention provides Hemostatic Patch F which comprises vasoconstrictor-nanoparticles, wherein at least one vasoconstrictor, e.g., (selected from any of the vasoconstrictors of the foregoing list numbered 1-12), is either encapsulated or immobilized by a bioabsorbable polymer, and wherein said bioabsorbable polymer,(e.g., having any of the characteristics selected from the foregoing list a.- j), comprises chitosan having any of the following characteristics:

k. The chitosan is derived from fungus, e.g. mushroom or mold;

l. The chitosan is derived from fungus, wherein the fungus is an Aspergillus, e.g., Aspergillus niger;

m. The chitosan is derived from mushroom, wherein the mushroom is an Agaricus, e.g., Agaricus bisporus;

n. Any of the foregoing wherein the chitosan has a molecular weight range of about Mv 30,000-220,000;

o. Any of the foregoing wherein wherein the chitosan a molecular weight range of about Mv 30,000-60,000;

p. Any of the foregoing wherein the chitosan has a range of apparent viscosity (e.g. at 1% solution in 1% acetic acid) of about <20 mPa·s to 90 (+/−30 mPa·s); e.g. <20 mPa·s, 40 (+/−20 mPa·s), 55 (+/−25 mPa·s), 90 (+/−30 mPa·s);

q. Any of the foregoing wherein the chitosan has a degree of acetylation (% mol) in a range of about 10%-40%;

r. Any of the foregoing wherein the chitosan has a degree of acetylation (% mol) in a range of about e.g, 10%-20%, 15%-25%, 20%-30%, or 30%-40%.

s. Any of the foregoing wherein the vasoconstrictor is encapsulated within the chitosan, and wherein there is a greater ratio of chitosan present in the nanoparticle relative to the amount of PLGA, e.g. a relative ratio amount of 80/20, chitosan to PLGA, (e.g., % w/w 80/20, chitosan to PLGA)

The above measurements may be carried out by any means known in the art. For example, it is contemplated that the viscosity of chitosan solutions may be measured at room temperature using a Brookfield type digital viscometer, e.g., DV-11+Pro. In another example, it is contemplated that the viscosity may be measured using a Ubbelohde type viscometer. In such an example, it is contemplated that the viscometer could be connected to a visco-clock to record the time of the passing solution.

In one aspect, the present invention provides Hemostatic Patch G which comprises a nanoparticle comprising chitosan, e.g., having any of the characteristics of foregoing list a.-s., and PLGA, wherein the relative ratio of chitosan to PLGA may be altered to adjust the release of the active ingredient, e.g. a vasoconstrictor of foregoing list 1-12. Without being bound by theory, it is believed that chitosan is hydrophilic. Therefore, where the active ingredient is hydrophilic (e.g. NPY or epinephrine) the addition of more chitosan relative to PLGA may result in a nanoparticle wherein the active ingredient is quickly released upon application or administration of the patch comprising the vasoconstrictor-nanoparticles, e.g., a relative ratio amount of 80/20, (e.g., % w/w 80/20, chitosan to PLGA) chitosan to PLGA, or a relative ratio amount of 90/10 (e.g., % w/w 90/10, chitosan to PLGA) chitosan to PLGA. Again, without being bound by theory, the addition of more PLGA, relative to the amount of chitosan, may result in a nanoparticle wherein the active ingredient is more slowly released, e.g., a relative ratio of 20/80 chitosan to PLGA (e.g., % w/w 20/80, chitosan to PLGA), or 10/90 chitosan to PLGA (e.g., % w/w 10/90, chitosan to PLGA).

In one specific example, the present invention provides Hemostatic Patch H which comprises NPY-nanoparticles, e.g., having any of the characteristics of foregoing list a.-s., wherein the NPY is encapsulated by chitosan, e.g., having any of the characteristics of foregoing list a.-s. In another specific example, the hemostatic patch comprises epinephrine-nanoparticles, e.g., having any of the characteristics of foregoing list a.-s., wherein the epinephrine is encapsulated by chitosan, e.g., having any of the characteristics of foregoing list a.-s.

In one aspect, the present invention provides Hemostatic Patch I which comprises any of the foregoing vasoconstrictors, e.g. of foregoing list 1-12, may be encapsulated within the nanoparticle. In another aspect, any of the foregoing vasoconstrictors, e.g. any of the foregoing list 1-12, may be immobilized by the nanoparticle. In one specific example the hemostatic patch comprises NPY-nanoparticles, wherein the NPY is covalently bound to the nanoparticle, e.g. having any of the characteristics of foregoing lists a.-s.

Methods of making chitosan nanoparticles may be found in the Applicants' own publications US 2011/0142947 and WO/2011/159899, the contents of each of which are incorporated herein by reference in their entireties. Similar chemical synthesis and methods of manufacture could be used to make and/or produce chitosan nanoparticles as described herein.

In one aspect, the present invention provides Hemostatic Patch J which comprises vasoconstrictor nanoparticles, wherein at least one vasoconstrictor e.g., selected from any of the foregoing numbered list 1.-12., is encapsulated or immobilized on a bioabsorbable polymer, e.g., having any of the characteristic of foregoing lists a.-s., and a vasoconstrictor. In one aspect said bioabsorbable polymer comprises chitosan, e.g. having any of the characteristics of foregoing lists a.-s., wherein the materials used to form the scaffold of the hemostatic patch may be formed from a natural material. The natural material from which the fibers are formed may be bioabsorbable, e.g., collagen, starch, gelatin, alginates, and/or dextrans.

In one aspect, the present invention provides Hemostatic patch K which comprises vasoconstrictor nanoparticles, wherein the vasoconstrictor is NPY, and the bioabsorbable polymer, e.g. having any of the characteristics of foregoing list a.-s., comprises chitosan, e.g., having any of the characteristics listed in a.-s., and a scaffold, wherein NPY is encapsulated within the chitosan.

In one aspect, the present invention provides Hemostatic patch L which comprises vasoconstrictor nanoparticles, wherein the vasoconstrictor is epinephrine, and the bioabsorbable polymer, e.g. having any of the characteristics of foregoing list a.-s., comprises chitosan, e.g., having any of the characteristics listed in a.-s., and a scaffold, wherein epinephrine is encapsulated within the chitosan.

In one aspect it is contemplated that the vasocontrictor-nanoparticles as described throughout, e.g., any Hemostatic Patch of A-L, may be applied to the scaffold of the hemostatic patch by any means known in the art. In one example the vasoconstrictor nanoparticles are applied via spray coating and/or painting the nanoparticles to the scaffold or matrix of the hemostatic patch. Such methods may be generally known in the art.

Without being bound by any theory, it is contemplated that the chitosan, in addition to being very effective in aiding in clotting at the wound, could be present in a gel form which could facilitate linkage with another compound. Moreover, without being bound by any theory, it is contemplated that the various free amino groups possessed by chitosan could allow it to be easily linked with other compounds.

In one aspect the present invention provides for method AA of using the hemostatic patch, e.g. any of Hemostatic Patch A-L, wherein the hemostatic patch comprises an effective amount of a formulation of vasoconstrictor nanoparticles, as described in any of Hemostatic Patch A-L, wherein said formulation of vasoconstrictor nanoparticles is sufficient to arrest bleeding in a patient in need thereof.

Method of method AA wherein the hemostatic patch comprises an effective amount of a formulation of a vasconstrictor nanoparticle, e.g, as described in any of Hemostatic Patch A-L wherein the vasoconstrictor is NPY.

Method of method AA wherein the hemostatic patch comprises an effective amount of a formulation of a vasconstrictor nanoparticle, e.g, as described in any of Hemostatic Patch A-L wherein the vasoconstrictor is epinephrine.

In one aspect, the present invention provides for methods of using the hemostatic patch, e.g. as described throughout, e.g., any Hemostatic Patch of A-L, to control or arrest bleeding internally. For example, one advantage of the the present invention described herein, e.g., any Hemostatic Patch of A-L, is that it can be used to control or arrest internal organ or tissue bleeding directly at the site of bleeding or trauma to the organ or tissue. In addition, the present invention also contemplates that the hemostatic patch may be used to control or arrest bleeding caused by damage to arteries. It is contemplated by the present invention that the arterial damage could have been caused by various catheterization procedures.

In one aspect the present invention described herein, e.g., any Hemostatic Patch of A-L, may be placed directly at the source of bleeding. In one aspect of the present invention the internal bleeding can be detected via endoscopic means. It is an advantage of the present invention that the hemostatic patch may be endoscopically placed on a wound in order to control or arrest bleeding in a patient.

In one aspect, the present invention described herein, e.g., any Hemostatic Patch of A-L, may be in the form of a spray or gel, or any other scaffold known in the art, and may be used during open, endoscopic or catheter-based procedures or surgeries. Patch compositions may be in the form of a spray or gel and include polyethylene glycol-based systems. It is contemplated that the vasocontrictant compound may be crosslinked to the gel. In one aspect the patch may be applied as a composition that forms a crosslinked gel when placed on the intended application site.

The present invention also provides methods for treating bleeding, e.g. arterial damage, internal organ damage, comprising placing a hemostatic patch as described herein, e.g., any Hemostatic Patch of A-L, over the injured area wherein said hemostatic patch comprises an effective amount of a mucoadhesive and an effective amount of a compound that causes vasoconstriction.

In one aspect, the present invention provides for a method for treating bleeding, e.g. arterial damage, internal organ damage, comprising placing a hemostatic patch as described herein, e.g., Hemostatic Patch A-L, over the injured area. In one aspect, hemostatic patch as described herein, e.g., any Hemostatic Patch of A-L, comprises nanoparticles which comprise chitosan and Neuropeptide Y. In another aspect the hemostatic patch comprises chitosan and epinephrine.

In one aspect the present invention described herein, e.g., any Hemostatic Patch of A-L, may be used to treat external patient bleeding as well.

It is contemplated that the present invention described herein, e.g., any Hemostatic Patch of A-L, may include any type of medical compress, patch, sponge, pad, swab, dressing, gel, and/or soluble matrix. It is further contemplated that any of these items may be applied using endoscopic means.

In another aspect, the present invention described herein, e.g., any Hemostatic Patch of A-L, of the present invention may be utilized to treat internal bleeding in a variety of different organs, the wounds or injury detected possibly endoscopically, for example:

s.) liver

t.) gall bladder

u.) urinary bladder

v.) ureter

w.) esophagus

x.) colon

y.) lung

z.) intestines

aa.)stomach and/or gastrointestinal system

bb.) gastrointestinal tract

In one aspect the hemostatic patch of the present invention, e.g., any Hemostatic Patch of A-L, may be utilized to control or arrest bleeding in those injuries caused by catheterization. In one example, the hemostatic patch can be used to repair arteries damaged by catheterization techniques. The hemostatic patch can be used to control or arrest arterial bleeding that is difficult or impossible to control by way of endoscopic techniques.

In another aspect, it is contemplated that the present invention described herein, e.g., any Hemostatic Patch of A-L, may be applied using certain surgical procedures, e.g., endoscopic procedures, laparoscopic procedures, arthroscopic procedures, and endoluminal and/or transluminal placement of the patch at a surgical site.

For example, it is contemplated that endoscopic procedures may include procedures to help aid bleeding caused by, e.g., vascular malformations, watermelon stomach, gastric antral vascular ectasias, radiation injury, benign neoplasms, post-polypectomy bleeding, post-endoscopic ampullary sphincterotomy bleeding, ulcers, Dieulafoy's lesions, benign or malignant neoplasms, Barrett's esophagus with or without dysplasia, varices, bleeding Mallory-Weiss tears, as well as to ablate malignant or hemorrhagic neoplasms. Additionally, it may be used to abate bleeding from portal hypertensive gastropathy or colitis, or for fistula occlusion. With respect to urology, the present invention described herein, e.g., any Hemostatic Patch of A-L, may be useful to treat, for example, chronic bleeding associated with retropubic prostatectomy, transurethral resection of the prostate, and other complications associated with urogenital surgical procedures. With respect to gynecology, the subject invention may be useful in treating lesions in the endocervical canal, such as uterine polyps. It is contemplated that the hemostatic patch can be applied to a bleeding site via colonoscopy. It is further contemplated that the patch may be applied to bleeding diverticulitis, to a biopsy site, to an arteriovenous malformation, malignant lesion, and/or bleeding hemorrhoids, in order to obtain hemostasis.

It is contemplated that the present invetion described herein, e.g., any Hemostatic Patch of A-L, may be used in cases where a patient has been diagnosed, or has potential symptoms of, hematuria. It is contemplated that where a patient has been diagnosed, or has potential symptoms of, hematuria that the hemostatic patch described herein may be used to obtain hemostasis or to arrest bleeding.

It is further contemplated that the hemostatic patch as described herein, e.g., e.g., any Hemostatic Patch of A-L, may be used to arrest bleeding and/or obtain hemostasis in postpartum hemorrhages. Postpartum hemorrhages may include, e.g., uterine atony, trauma, retained placenta, and/or coagulopathy.

In one aspect the hemostatic patch described herein, e.g., any Hemostatic Patch selected of A-L, may be used during various dental procedures. It is contemplated that the patch may be used either to arrest bleeding that had been preexisiting, as well as bleeding that is caused by the dental procedure itself

In yet another aspect it is contemplated that the hemostatic patch described, e.g., any Hemostatic Patch of A-L, herein may be used to arrest bleeding and/or obtain hemostasis wherein a subject exhibits hemoptysis. Said hemoptysis may include, e.g., bronchitis or pneumonia. However, hemoptysis may also include, e.g., lung neoplasm, aspergiloma, tuberculosis, bronchiectasis, coccidiomycosis, pulmonary embolism, pneumonic plague, and cystic fibrosis. Said hemoptysis may also be the result of the administration of warfarin. It is contemplated that the hemostatic patch as described herein may be applied using a bronchoscopes.

In still another aspect, it is contemplated that the present invention described herein, e.g., any Hemostatic Patch of A-L, may used to treat external bleeding. In one aspect the hemostatic patch as described herein may be able to treat, e.g., war wounds, boxing lacerations, and/or dental procedures.

In still another aspect the present invention provides for the following methods of use directed toward the hemostatic patch. Method I which is a method of using a hemostatic patch as described herein, e.g., any Hemostatic Patch of A-L, comprising an effective amount of a mucoadhesive and a compound that causes vasoconstriction wherein the method comprises administering said hemostatic patch to a patient in need thereof and applying said hemostatic patch to the area of trauma or injury of said patient to thereby control or arrest bleeding in said patient.

1.1 The method of Method I, wherein said mucoadhesive is chitosan and said compound that causes vasoconstriction is Neuropeptide Y.

1.2 The method of Method I or 1.1, wherein said hemostatic patch is administered endoscopically.

1.3 The method of any of Method I, 1.1, or 1.2, wherein said hemostatic patch is applied to an internal organ or tissue to control or arrest bleeding.

1.4 The method of Method 1.3, wherein the hemostatic patch is applied to an internal organ selected from the group consisting of: liver, gall bladder, urinary bladder, ureter, esophagus, colon, lung, intestines, stomach and/or gastrointestinal system, and gastrointestinal tract.

1.5 The method of any of the preceding methods, wherein said hemostatic patch is applied to an artery to control or arrest bleeding.

1.6 The method of any of the preceding methods, wherein the arterial bleeding is caused initial by catheterization.

1.7 The method of any of the preceding methods, wherein the mucoadhesive and the compound that causes vasoconstriction are present in a stable matrix.

1.8 The method of method 1.7, wherein the mucoadhesive is chitosan and the compound that causes vasoconstriction is Neuropeptide Y.

1.9 The method of any of the preceding methods, wherein the mucoadhesive and the compound that causes vasoconstriction are covalently bound.

1.10 The method of method 1.10, wherein the mucoadhesive is chitosan and the compound that causes vasoconstriction is Neuropeptide Y.

1.11 The method of any of the preceding methods wherein the compound that cause vasoconstriction is epinephrine.

The present invention contemplates that the hemostatic patch as described herein, e.g. Hemostatic Patch of A-L, may be used in any method or any particular use as described herein. Such specific procedures, e.g., surgical methods, of using hemostatic patches may be known in the art.

Administration or application routes for the hemostatic patch described herein include, but are not limited to intravenous, intra-arterial, intracardiac, subcutaneous, intramuscular, orally, intrapulmonary (e.g., by inhalation), intradermal, topically, rectally, via catheter, or endoscopically. The formulation may be for immediate release, e.g., via intravenous, intra-arterial, or intracardiac injection, or may be in the form of a sustained release depot formulation, e.g., a hemostatic patch as described herein, e.g. any of Hemostatic Patch A-L, comprising a bioabsorbadable polymer comprising the vasoconstrictor nanoparticles of the invention, for example for subcutaneous or intramuscular application, resulting in release of vasoconstrictor over a period of days or weeks.

Various methods of synthesizing NPY-nanoparticles are provided. For example, a single emulsion process may produce chitosan-PLGA nanoparticles encapsulating NPY. In yet another example, a process involving gelation/conjugation of preformed biodegradable polymers produces 1) chitosan nanoparticles encapsulating NPY with and without glutaraldehyde as a cross-linker; or 2) chitosan-PLGA nanoparticles encapsulating NPY. Other cross-linkers may be used.

In yet another example, a process involving chemical bonding of NPY on the surface of chitosan-PLGA nanoparticles produces 1) chitosan-PLGA nanoparticles immobilizing NPY or 2) chitosan-PLGA nanoparticles immobilizing NPY and additionally including chitosan-PLGA nanoparticles encapsulating NPY.

For example, in one aspect, PLGA and NPY are first immersed in a 1% PVA solution and chitosan. They are then stirred and sonicated. Then a dialysis step is performed. After a dialysis step occurs, PLGA-chitosan nanoparticles encapsulating NPY are produced. Then in the final step, the nanoparticles may then have a chitosan layer cross-linked with glutaraldehyde. Other cross-linkers may be used.

In another aspect, PLGA and epinephrine are first immersed in a 1% PVA solution and chitosan. They are then stirred and sonicated. Then a dialysis step is performed. After a dialysis step occurs, PLGA-chitosan nanoparticles encapsulating epinephrine are produced. Then in the final step, the nanoparticles may then have a chitosan layer cross-linked with glutaraldehyde. Other cross-linkers may be used.

In one aspect, an entrapment efficiency may also be measured. For example, the entrapment efficiency may be calculated to be the total amount of NPY in the nanoparticles/initial concentration of NPY added to make the formulation ×100. In another aspect, similar calculations may be done for epinephrine.

### EXAMPLES

### Example 1

### Synthesis of NPY Encapsulated Nanoparticles

Chitosan nanoparticles encapsulating NPY are produced using a reverse micellar method. Chitosan polymer and NPY are added to 0.1M AOT/hexane (AOT-Aerosol OT is used as a surfactant) solution to form reverse micelles. Bifunctional reagent gluteraldehyde is added to this reverse micelles system as a cross-linking agent. The chemical cross-linking of chitosan polymers with gluteraldehyde occurs by Schiff's reaction of aldehyde groups on gluteraldehyde and amino groups on the chitosan chain. Finally nanoparticles are separated out by high speed centrifugation.

In these examples, nanoparticles are optimized as to size, and entrapment efficiency to get an optimum formulation with maximum loading.

### Example 2

### Synthesis of Chitosan-PLGA Nanoparticles

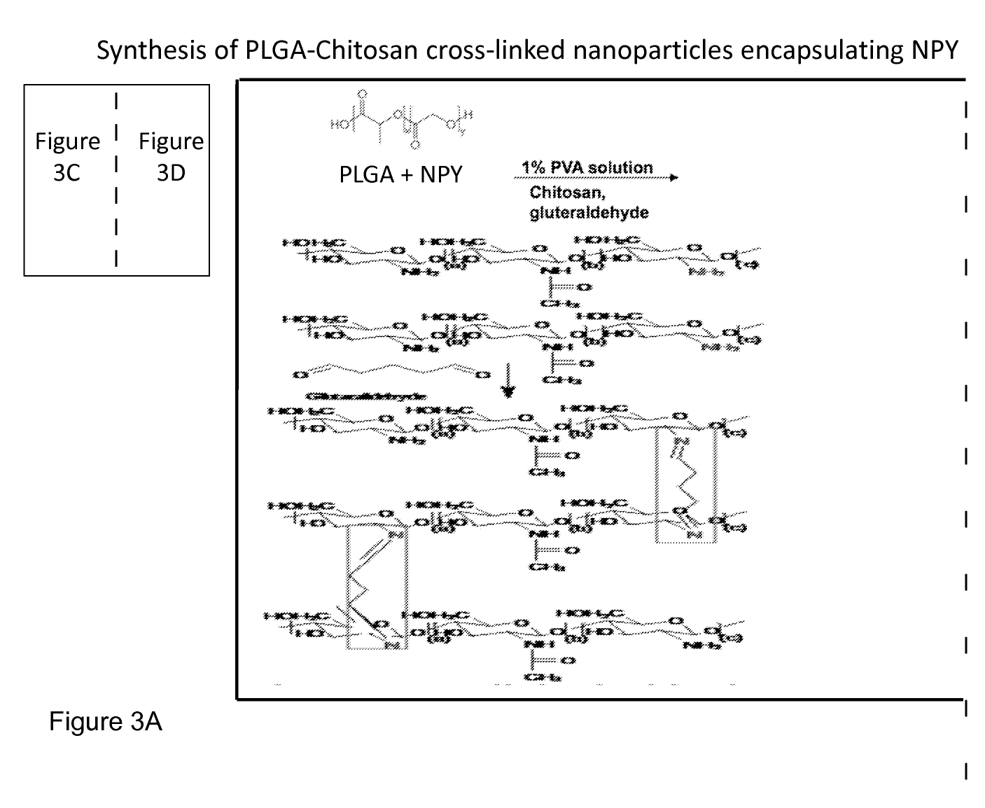

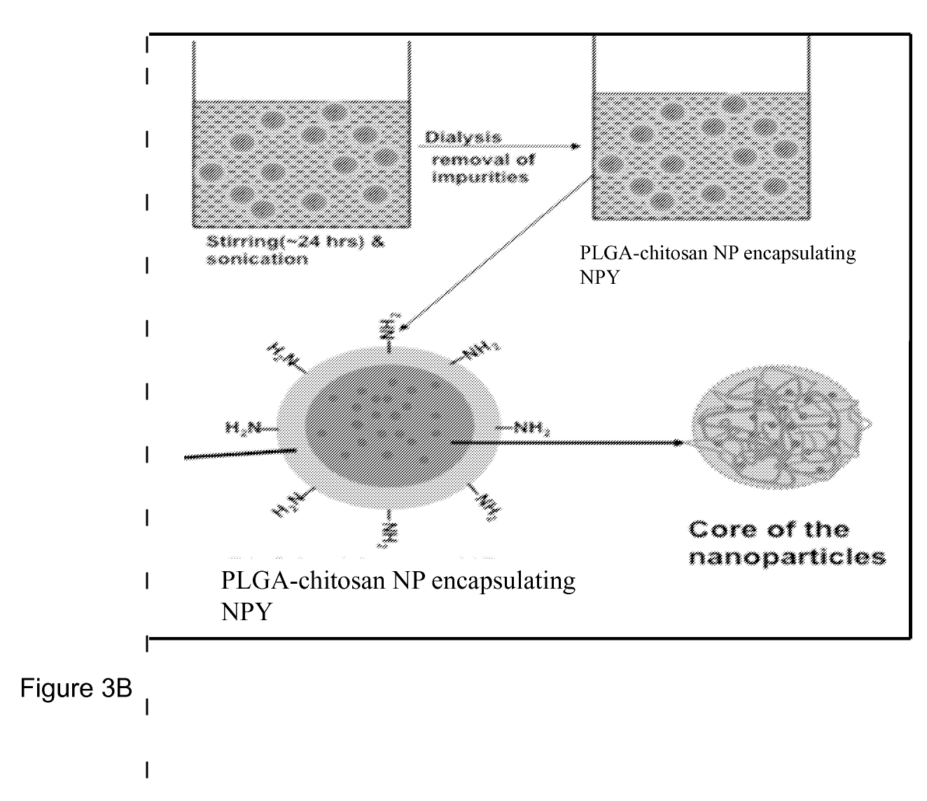

FIGS. 3A and 3B depict the synthesis and preparation of chitosan-PLGA hybrid nanoparticles with and without VIP. In FIG. 3A, PLGA is mixed with chitosan and PVA(1%) in an overnight stirring and sonication step. Subsequently the mixture undergoes a dialysis step to remove impurities. PVA is used as a stabilizer, while DMSO (0.1% v/v) and acetic acid (0.1% v/v) were incorporated as solvents. These may be removed by the subsequent dialysis step. FIG. 3B, PLGA, NPY, chitosan, and gluteraldehyde are mixed together, for approximately twenty-four hours, in a stirring and sonication step. Subsequently the mixture undergoes a dialysis step to remove impurities. The result is a PLGA-chitosan nanoparticle, wherein the chitosan layer is cross-linked with gluteraldehyde.

NPY encapsulated in nanoparticles with different degrees of cross-linking is tested for optimal pharmacokinetics. The formulation is optimized for loading efficiency. The ratios of different constituents are manipulated for optimum delayed release. To achieve that goal, the following parameters are evaluated: Particle size analysis by DLS spectroscopy, zeta potential measurement, in vitro release kinetics, Transmission Electron Microscopy for size confirmation, measurement of NPY inside the nanoparticles (by HPLC or LC/MS).

### Example 3

### Synthesis of Epinephrine Encapsulated Nanoparticles

Chitosan nanoparticles encapsulating epinepherine are produced using a reverse micellar method. Chitosan polymer and epinephrine are added to 0.1M AOT/hexane (AOT-Aerosol OT is used as a surfactant) solution to form reverse micelles. Bifunctional reagent gluteraldehyde is added to this reverse micelles system as a cross-linking agent. The chemical cross-linking of chitosan polymers with gluteraldehyde occurs by Schiff's reaction of aldehyde groups on gluteraldehyde and amino groups on the chitosan chain. Finally nanoparticles are separated out by high speed centrifugation.

In these examples, nanoparticles are optimized as to size, and entrapment efficiency to get an optimum formulation with maximum loading.

### Example 4

### Synthesis of Chitosan-PLGA Nanoparticles

PLGA is mixed with chitosan and PVA(1%) in an overnight stirring and sonication step. Subsequently the mixture undergoes a dialysis step to remove impurities. PVA is used as a stabilizer, while DMSO (0.1% v/v) and acetic acid (0.1% v/v) were incorporated as solvents. These may be removed by the subsequent dialysis step. PLGA, epinephrine, chitosan, and gluteraldehyde are mixed together, for approximately twenty-four hours, in a stirring and sonication step. Subsequently the mixture undergoes a dialysis step to remove impurities. The result is a PLGA-chitosan nanoparticle, wherein the chitosan layer is cross-linked with gluteraldehyde.

Epinephrine encapsulated in nanoparticles with different degrees of cross-linking is tested for optimal pharmacokinetics. The formulation is optimized for loading efficiency. The ratios of different constituents are manipulated for optimum delayed release. To achieve that goal, the following parameters are evaluated: Particle size analysis by DLS spectroscopy, zeta potential measurement, in vitro release kinetics, Transmission Electron Microscopy for size confirmation, measurement of NPY inside the nanoparticles (by HPLC or LC/MS).

### Example 5

### Synthesis of Hydrophobic Chitosan

Hydrophobic chitosan polymer is synthesized according to the following scheme:

To Chitosan (0.200 g) (75-85% deacetylated) solution in HCl (0.2 N, 20 mL), MeOH (20 mL), NHS, lithocholic acid (106.4 mg, 0.283 mmol) and pyridine (647.0 μL) are added. After overnight stirring at room temperature, another portion of Me0H (40 mL) is added to obtain a clearer reaction mixture. EDAC (81.2 mg, 0.424 mmol) is added and magnetically stirred at room temperature for 24 hrs. Chitosan product is precipitated out by ammonium hydroxide (3 mL) and collected by centrifugation. The precipitates are washed three times with deionized water. The precipitates are then redissolved in 1% AcOH (20 mL), washed with DCM:MeOH (1:4) (3×20 mL), precipitated again with ammonium hydroxide (3 mL), washed with deionized water (3×20 mL) and lyophilized for 48 hours.

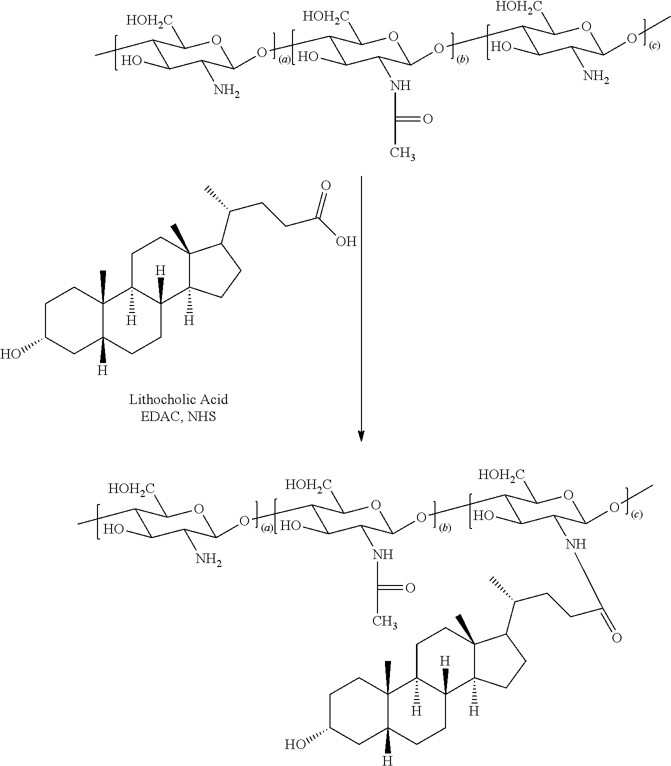

Different ratios of chitosan: lithocholic acid are synthesized.
The examples provided in the detailed description are merely examples, which should not be used to limit the scope of the claims in any claim construction or interpretation.

1.-9. (canceled)

10. A hemostatic patch, wherein the patch comprises a mucoadhesive and a formulation of vasoconstrictant nanoparticles, wherein the vasoconstrictant nanoparticle comprises a vasoconstrictor selected from, Neuropeptide Y, Neuropeptide Y analogues, epinephrine, norepinephrine, vasopressin, phenylephrine, pseudoephedrine, metaraminol, Peptide YY, Angiotensin II, Muscarinic agonists, Endothelin; and Andrenergic agonists, wherein the vasoconstrictor is encapsulated or immobilized on a bioabsorbable polymer.

11. The hemostatic patch of claim 10, wherein the bioabsorbable polymer comprises chitosan.

12. The hemostatic patch of claim 10, wherein the polymer comprises poly(lactic-co-glycolic acid) (PLGA).

13. The hemostatic patch of claim 10, wherein the polymer comprises chitosan crosslinked using glutaraldehyde.

14. The hemostatic patch of claim 10, wherein the polymer comprises chitosan crosslinked to PLGA.

15. The hemostatic patch of claim 10, wherein the vasoconstrictant nanoparticle comprises a vasoconstrictor selected from, Neuropeptide Y, Neuropeptide Y analogues, epinephrine, norepinephrine, vasopres sin, phenylephrine, pseudoephedrine, metaraminol, Peptide YY, Angiotensin II, Muscarinic agonists, Endothelin; and/or, Andrenergic agonists), and the vasoconstrictor is covalently bound to the polymer.

16. (canceled)

17. The hemostatic patch of claim 11, wherein the chitosan is derived from fungus.

18. The hemostatic patch of claim 11 wherein the fungus is an Aspergillus.

19. The hemostatic patch of claim 11, wherein the chitosan is derived from a mushroom, wherein the mushroom is an Agaricus.

20. The hemostatic patch of claim 11, wherein the chitosan has a molecular weight range of about My 30,000-60,000.

21. The hemostatic patch of claim 11, wherein the chitosan has a range of apparent viscosity of about <20 mPa·s to 90 (+/−30 mPa·s).

22. The hemostatic patch of claim 11, wherein the chitosan has a degree of acetylation (% mol) in a range of about 10%-40%.

23. The method of treatment using a hemostatic patch of claim 10, wherein the hemostatic patch comprises an effective amount of a mucoadhesive and a compound that causes vasoconstriction, Neuropeptide Y, Neuropeptide Y analogues, epinephrine, norepinephrine, vasopressin, phenylephrine, pseudoephedrine, metaraminol, Peptide YY, Angiotensin II, Muscarinic agonists, Endothelin; and, Andrenergic agonists, wherein the method comprises administering said hemostatic patch to a patient in need thereof and applying said hemostatic patch to the area of trauma or injury to thereby control or arrest bleeding.

24. A method of making a vasoconstrictant nanoparticle to be used in a hemostatic patch, comprising:
providing PLGA and a vasoconstrictor; e.g. selected from any of the following, Neuropeptide Y, Neuropeptide Y analogues, epinephrine, norepinephrine, vasopressin, phenylephrine, pseudoephedrine, metaraminol, Peptide YY, Angiotensin II, Muscarinic agonists, Endothelin; and, Andrenergic agonists immersing the PLGA and a vasoconstrictor in a 1% solution including chitosan;
stirring and sonicating; and
performing a dialysis step, to yield the a vasoconstrictor-nanoparticle.

25.-34. (canceled)
